SEQUENCE LISTING (2 sequences)

SEQ ID NO: 1 - DNA, 77 bp, Artificial Sequence
  Synthesized Forward Primer
gatccccagccctcatcttctactacttcattcaagagatgaagtagtagaagatgagggctggttttttagatcta

SEQ ID NO: 2 - DNA, 77 bp, Artificial Sequence
  Synthesized Reverse Primer
agcttagatctaaaaaaccagccctcatcttctactacttcatctcttgaatgaagtagtagaagatgagggctggg

(19) United States
(12) Patent Application Publication
(10) Pub. No.: US 2020/0179430 A1
(43) Pub. Date: Jun. 11, 2020

(54) USE OF P2Y1 RECEPTOR AND ANTAGONIST THEREOF IN PREVENTION AND TREATMENT OF DEPRESSION AND/OR ANXIETY DISORDER

(71) Applicant: SOUTHERN MEDICAL UNIVERSITY, Guangdong (CN)

(72) Inventors: Tianming GAO, Guangdong (CN); Qian WANG, Guangdong (CN); Ying KONG, Guangdong (CN); Dingyu WU, Guangdong (CN); Jianming YANG, Guangdong (CN)

(73) Assignee: SOUTHERN MEDICAL UNIVERSITY, Guangdong (CN)

(21) Appl. No.: 16/631,454

(22) PCT Filed: Oct. 23, 2017
(86) PCT No.: PCT/CN2017/107289

§ 371 Date: Jan. 16, 2020

(30) Foreign Application Priority Data
Aug. 21, 2017 (CN) 201710719175.1

(51) Int. Cl. A61K 31/7076; A61P 25/24; G01N 33/68

(52) U.S. Cl. CPC A61K 31/7076; G01N 2500/04; G01N 33/6896; A61P 25/24

(57) ABSTRACT

P2Y1 receptor and an antagonist thereof are used in anti-depression and/or anti-anxiety disorder. It has been verified through experimental studies that the P2Y1 receptor can be used for screening for a medicine of anti-depression and/or anti-anxiety disorder, and also used for screening for depression and/or anxiety disorder, and for early warning of depression and/or anxiety disorder; and the antagonist of P2Y1 receptor has anti-depression and anti-anxiety effects which can be used to prepare a medicine of anti-depression and/or anti-anxiety disorder.

### BACKGROUND

### Technical Field

The present invention relates to the field of biological pharmaceutical and medical technology, and specifically relates to use of P2Y1 receptor as a target in screening for anti-depression and/or anti-anxiety disorder and a medicine thereof, or use of an antagonist for P2Y1 receptor in preparing a medicine of anti-depression and/or anti-anxiety disorder.

### Description of Related Art

Depression is the most common mood disorder, which is severely threatening the mental and physical health of human. Depression is a kind of mood disorder having main clinical features such as low mood, damaged cognitive function and decline in volitional activity, and characteristics such as high incidence, high disability rate, heavy burden and the like. According to World Health Organization, the incidence is approximately 15% to 17% and appears to be an increasing tendency. It is predicted that depression will be the leading disability cause globally in 2020. Depression not only severely threatens individual health and life of the patient, but also brings huge economic and life burdens to the society and the family. Therefore, prevention and treatment of depression has become a significant public health issue.

Current treatment of depression mainly relies on the medical treatment, but still faces several serious challenges: first, drug efficiency is low that about 35% of the patients are insensitive to the current antidepressants; second, drug compliance is poor that it is slow for the current antidepressants to take effect which require at least 3 to 6 weeks, and many patients would give up halfway; and third, the medicines have strong side effect and recurrence rate is high after drug withdrawal. Therefore, finding an effective target and researching on a new-type medicine that works fast with long-lasting effect and fewer side effects have become the problems to be urgently solved.

Although anxiety disorder and depression are two different kinds of mental disorders, they are in close relationship and present in combination usually. Current research indicates that the anxiety disorder shares similar symptoms with the depression; they are the same response to the pathogenic factor but have characteristic symptoms varying with each individual. It is also believed that with aggravation and migration of the disease conditions, either one can develop secondary symptoms of the other. Due to so many similarities, it is difficult to identify these two diseases. Therefore, a combined therapy for these two diseases is recommended.

Adenosine triphosphate, also called ATP, is the most direct energy source in the life entity and is also recognized as an important neurotransmitter of which corresponding receptor exists extensively in the life entity. The receptors of ATP are called P2 receptors, which are classified as the gated ion channel P2X receptors family and the G protein-coupled P2Y receptors family. When the P2X receptors are ion channel receptors, the channel can be opened during activation to alter the ionic concentration at either side and exert a signal transmission effect. When the P2Y receptors are G protein-coupled receptor, different subtypes of P2Y receptors activate different intracellular signal transduction pathways by the G protein to execute a specific physiological function. The P2Y receptors have eight subtypes in mammals: P2Y1, P2Y2, P2Y4, P2Y6, P2Y11, P2Y12, P2Y13 and P2Y14 respectively, which consist of 308 to 379 amino acids. As a typical G protein, the P2Y receptor includes 7 transmembrane domains with the extracellular N-terminal and the intracellular C-terminal.

The P2Y receptors are widespread, and have relatively high distribution density in the nervous system and expression in both neurons and glial cells. The P2Y1, P2Y2, P2Y4 and P2Y12 receptors each express in the neurons of cerebral cortex, hippocampus and cerebellar cortex. Purine neurotransmitters such as ATP, ADP, adenosine and the like can regulate excitability of the neurons and synaptic transmission between the neurons by means of different P2Y receptors, so as to regulate and control local activity in brain circuits. In the glial cells (astrocytes, oligodendrocytes, microglias), a large amount of P2Y receptors express and participate in the neuron-glial cell interaction. It has been reported by the research that the P2Y receptors participate in neurological pathological pain and plays a key role in the generation and duration of pain sense, which is expected to be a new therapeutic target in the treatment. It has also been reported that the P2Y receptor participates in neurological degenerative disease, wherein the P2Y1 receptor may directly participate in the pathological process of neurological degenerative disease. It has been further reported that the P2Y receptor participates in the activation of glial cells in various manners during the brain injury and inhibits the activity of glial cells at the early stage of disease, and may become an acting site of medicines for treating epilepsy, chronic pain, brain tumor and the like in the future.

In the aspect of the current clinical application, an agonist and an antagonist of the P2Y1 receptor are mainly used in coagulation of platelet in the cardiovascular system and treatment of the immune-inflammatory injury.

### SUMMARY

The objective of the present invention is to overcome the above-mentioned deficiencies existing in the medicines for treating depression and anxiety disorder in the prior art, and to provide use of an antagonist of P2Y1 receptor in treating depression and/or anxiety disorder, and use of the P2Y1 receptor as a target in screening for depression and/or anxiety disorder and a medicine thereof.

In order to determine the overall level effect of the P2Y1 receptor, P2Y1 receptor conventional knockout mice were introduced in the present invention from JAX, US. Upon verification, the P2Y1 receptor no longer expressed in the conventional knockout mice (as shown in FIG. 4). In the forced swimming test, the P2Y1 receptor conventional knockout mice had significantly decreased immobility time with respect to the P2Y1 wild type mice, and expressed anti-depression-like behavior (as shown in FIG. 5). In the tail suspension test, the P2Y1 receptor conventional knockout mice also had significantly decreased immobility time with respect to the P2Y1 wild type mice, and displayed anti-depression-like behavior (as shown in FIG. 6). The above results further demonstrate that blocking the P2Y1 receptor can exert an anti-depression effect. Thus, an antagonist of P2Y1 receptor can be used for anti-depression and/or anti-anxiety disorder.

As a further preferably technical solution of the present invention, the antagonist of P2Y1 receptor is a compound or polypeptide inhibiting the P2Y1 receptor.

In order to further verify whether the P2Y1 receptor can be used as a target of anti-depression and/or anti-anxiety, and to avoid the compensatory effect that may exist in the conventional knockout mice, P2Y1 receptor specifically-interfering adeno-associated virus was prepared in the present invention to be observed whether the virus affects the depression-like behavior. The mice were intracerebrally injected with a trace amount of the P2Y1 receptor-interfering RNA adeno-associated virus at the ventral tegmental area, and then a forced swimming test was carried out. It is found from the results that with respect to the control group mice, the P2Y1 receptor-interfering RNA in the experimental group mice significantly decreased the expression of P2Y1 receptor. In the forced swimming test, the experimental group mice had obviously decreased immobility time and obvious anti-depression effect (as shown in FIG. 7 and FIG. 8). The inventors further used a depression model, chronic social defeat stress model to verify the effect of the P2Y1 receptor specifically-interfering adeno-associated virus. After modeling of the depression model was completed, the mice took a day off, and then were administered the P2Y1 receptor specifically-interfering adeno-associated virus, and two weeks later the mice were subjected to a behavior test. In the social interaction test, situation of the social defeat depressed mice was reversed (as shown in FIG. 9). In the forced swimming test, the immobility time of the social defeat depressed mice which was increasing originally returned to the level of the control group (as shown in FIG. 10). In the sucrose preference test, a decreased sucrose preference of the social failure depressed mice was reversed (as shown in FIG. 11). The above results demonstrate that the P2Y1 receptor is blocked by the antagonist, or lowering the expression level of the P2Y1 receptor can exert an anti-depression effect. Thus, the P2Y1 receptor can be used as a target for screening for medicines and clinical diagnosis reagents for preventing and treating anti-depression and/or anti-anxiety disorder, and also as a biomarker for screening for depression and/or anxiety disorder, and for early warning of depression and/or anxiety disorder.

It is found by the pharmacodynamic and pharmacology experiments in the present invention that: specific antagonists of P2Y1 receptor, MRS2500 and derivatives thereof, and MRS2179 and derivatives thereof can effectively improve depressive situation of the experimental mice. In order to test the anti-depression effect of the specific antagonist MRS2500 of the P2Y1 receptor, 2.5 mg/kg MRS2500 was administered intraperitoneally to the mice. 30 minutes later, the mice were subjected to the forced swimming test for 6 minutes, and immobility time within the last 4 minutes was determined to evaluate the effect of the antidepressant. It is found from the result that administration of MRS2500 shows the same anti-depression behavior as administration of the conventional antidepressant Fluoxetine (as shown in FIG. 1). The main pathogenesis of depression is deemed to be caused by neurological abnormalities such as neuroendocrine and nerve regeneration. Thus, in order to further test the effect of the antidepressant, in the present invention, the mice were administered 0.1 μM MRS2500 and 0.2 μM MRS2179 via implanted tube at the lateral ventricle, and 30 minutes later, the mice were subjected to a forced swimming test for 6 minutes. Compared with the control group, MRS2500 and MRS2179 significantly reduce the immobility time of the mice during forced swimming, which demonstrates that the antagonist of P2Y1 receptor exhibits an anti-depression effect (as shown in FIG. 2).

In order to further test the anti-depression effect of the antagonist of P2Y1 receptor, a chronic social defeat stress model was used to determine the anti-depression effect of the antagonist for P2Y1 receptor in the present invention. As invaders, C57BL/6J mice were subjected to physical aggression for 10 minutes daily from CD1 mice which were raised individually for a long term. After physical contact, they were separated by a transparent and perforate organic glass sheet. The C57BL/6J mice were subjected to visual and olfactory stresses for 24 hours, and after 10 consecutive days, modeling was accomplished. A tube was implanted to the lateral ventricle. After a single dose of administration, situation of the mice with depressive situation in the MRS2500 group was significantly improved. The above results further demonstrate that upon blocking the P2Y1 receptor, MRS2500 has an anti-depression effect (as shown in FIG. 3).

In conclusion, the antagonist of P2Y1 receptor of the present invention can be used to prepare a medicine of anti-depression and/or anti-anxiety disorder, which includes an effective dosage of the antagonist of P2Y1 receptor and a pharmaceutically acceptable carrier.

Preferably, the antagonist of P2Y1 receptor is a compound or a polypeptide inhibiting the P2Y1 receptor.

More preferably, the antagonist of P2Y1 receptor is MRS2179 and derivatives thereof, or MRS2500 and derivatives thereof.

When the antagonist of P2Y1 receptor of the present invention is used to prepare a medicine of anti-depression and/or anti-anxiety disorder, a preferred dosage form of the medicine is liquid or solid. Particularly, the liquid dosage form can be injection, solution, suspension, emulsion or aerosol; and the solid dosage form can be tablet, capsule, pill, powder-injection, sustained release preparation or various particulate drug delivery systems.

Compared to the prior art, the present invention has the following advantages and beneficial effects.

The present invention provides a target P2Y1 receptor relative to depression and anxiety disorder, and an antagonist of the P2Y1 receptor which is used to prepare a medicine of anti-depression and/or anti-anxiety disorder. The present invention provides a new target and medicine for diagnosis and treatment of depression and anxiety disorder, and has good application prospect and research value.

### BRIEF DESCRIPTION OF THE DRAWINGS

FIG. 1 is a result diagram of a forced swimming test of C57BL/6J mice after intraperitoneal administration of MRS2500;

FIG. 2 is a result diagram of a forced swimming test of C57BL/6J mice after lateral ventricle administration of MRS2500 and MRS2179;

FIG. 3 is a result diagram of a forced swimming test of mice modeled by chronic social defeat stress model after administration of MRS2500;

FIG. 4 is a result diagram of a P2Y1 receptor protein expression level test of P2Y1 receptor conventional knockout mice;

FIG. 5 is a result diagram of a forced swimming test of the P2Y1 receptor conventional knockout mice;

FIG. 6 is a result diagram of a tail suspension test of the P2Y1 receptor conventional knockout mice;

FIG. 7 is a location diagram of interfering RNA adeno-associated virus which was intracerebrally injected to the ventral tegmental area of C57BL/6J mice;

FIG. 8 is a result diagram of a forced swimming test of the C57BL/6J mice intracerebrally injected with the interfering RNA adeno-associated virus at the ventral tegmental area of mice;

FIG. 9 is a result diagram of a social interaction test of mice modeled by chronic social defeat stress model after administration of P2Y1shRNA;

FIG. 10 is a result diagram of a forced swimming test of mice modeled by the chronic social defeat stress model after administration of P2Y1shRNA;

FIG. 11 is a result diagram of a sucrose preference test of mice modeled by the chronic social defeat stress model after administration of P2Y1shRNA.

### DESCRIPTION OF THE EMBODIMENTS

In order to make the objectives, technical solutions and beneficial effects of the present invention more clear, the present invention is further described in detail below in combination with following embodiments. It should be understood that the embodiments described in the present specification are merely to illustrate the present invention which is not limited thereto, and parameters and ratios of the embodiments can be selected according to circumstances and make no substantial effect on the results.

### Embodiment 1 Anti-Depression and Anti-Anxiety Pharmacologic Experimental Studies on Specific Antagonists of P2Y1receptor, MRS2500 and MRS2179

1. Forced Swimming Test (FST):

Forced swimming test which was proposed by Porsolt in 1977 was applied to the rats at the earliest, and subsequently extended to the mice. The forced swimming test is used to screening for antidepressants which are found to decrease immobility time of the forced swimming mice, and such model is one of the most common-used models for screening for antidepressants at present. The test steps are as follows: experimental mice adapted in an ethology laboratory for at least 60 minutes in advance, and the following test was carried out: an individual mouse was put in a cylinder glass jar (height: 45 cm; diameter: 19 cm) with a water depth of 23 cm at water temperature of 22° C.-25° C., and was subjected to the forced swimming for 6 minutes. Accumulative immobility time within the last 4 minutes was recorded (mainly behaves as stopping struggling, floating upright, or having only small limb movements to keep the head emerge from the water) as an index of tested depression-like behavior. Effect of the antidepressant can shorten the immobility time of the experimental mice.

Experimental animals: C57BL/6J mice, grade SPF, male, body weight of 20 to 24 g. The animals were provided by the Laboratory Animal Center of Southern Medical University (Guangzhou, China). The mice were put in the SPF grade animal feeding room for feeding in separate cages, with 3 to 4 mice in each cage. The plastic cage has a size of 300 mm×170 mm×120 mm. Room temperature: 24±1° C., light-dark cycle: 12 hours/12 hours, illumination time: 7:00 a.m. to 7:00 p.m., relative humidity: 70% to 85%. The indoor environment was kept quiet and the mice could eat and drink freely. The tests were carried out at 1:00 to 5:00 in the afternoon. Before the test, the mice entered the experimental environment in advance for adaptation and were touched by the experimental personnel for adaptation.

In the forced swimming test of the C57BL/6J mice, the animals were divided into 5 groups randomly with 10 mice in each group. The animals in each group were surgically implanted with a medical perfusion tube at the lateral ventricle and fed for seven days for recovery. Then, four groups of animals were subjected to the following treatment respectively: by using a microdose drug delivery system, administering the corresponding reagent: a control solution (artificial cerebrospinal fluid, 1 μL, 1 μL/min), MRS2500 (0.1 μM, 1 μL, 1 μL/min), MRS2179 (0.2 μM, 1 μL, 1 μL/min). After the injection for each group was completed, the forced swimming test was carried out 10 minutes later.

2.5 mg/kg MRS2500 was intraperitoneally administered to the mice, and 30 minutes later, the mice were subjected to the forced swimming test for 6 minutes. Immobility time of swimming within the last 4 minutes was determined to evaluate the effect of the antidepressant.

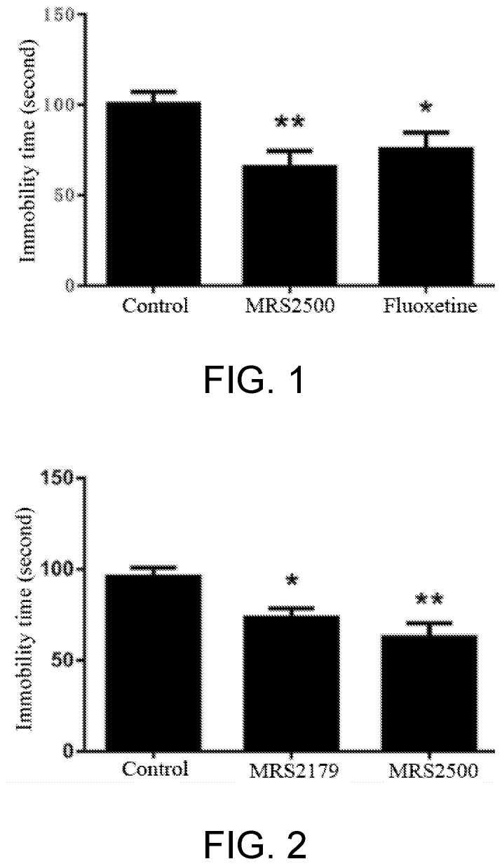

Test result: administration of MRS2500 shows the same anti-depression like behavior as administration of the conventional antidepressant Fluoxetine (as shown in FIG. 1).

As shown in FIG. 2, both administration of MRS2500 and administration of MRS2179 to the C57BL/6J mice at the lateral ventricle can significantly shorten the immobility time of swimming for the mice, indicating that the antagonist of P2Y1 receptor has a clear anti-depression effect.

2. Social Failure Model:

The chronic social defeat stress model was carried out referring to the Golden method.

The C57BL/6J mice were randomly divided into a control group and a stress group. The C57BL/6J mouse in the stress group was daily put in a cage with a screened CD-1 mouse therein, for 10-minute body contact. As an invader, the C57BL/6J mouse behaved as escape, fear, obedience and the like after being attacked. After the contact, within the 24 hours after they were separated by a transparent and perforate partition, the C57BL/6J mouse could smell, see and hear the CD-1 mouse at the opposite side for mental stress. The C57BL/6J mice took turns daily to guarantee to be subjected to even stress. The test was continued for ten days. The CD-1 mouse in the control group was replaced by another C57BL/6J mouse, and took turns daily, but body contact was avoided. After modeling, the mice were fed in separate cages and were subjected to tube-implant surgery at the lateral ventricle. The control group and the test group were administered the corresponding reagent respectively: a control solution (artificial cerebrospinal fluid, 1 μL, 1 μL/min), MRS2500 (0.1 μM, 1 μL, 1 μL/min). After administration, depression level of the mice was tested by a social interaction test. The social interaction test was divided into two stages, 2.5 minutes for each stage. First stage (no target): the CD-1 mouse was not put in a transparent and perforate organic glass box; the C57BL/6J mice were put at the distal side, and dwell durations thereof at the interaction area and at the corner within 2.5 minutes were recorded. Second stage (target): the CD-1 mouse was put in the transparent and perforate organic glass box; the C57BL/6J mice were put at the distal side, and dwell durations thereof at the interaction area and at the corner within 2.5 minutes were recorded.

Experimental animals: C57BL/6J mice, grade SPF, male, body weight of 20 to 24 g. The animals were provided by the Laboratory Animal Center of Southern Medical University (Guangzhou, China). The mice were put in the SPF grade animal feeding room for feeding in separate cages, with 3 to 4 mice in each cage. The plastic cage has a size of 300 mm×170 mm×120 mm. Room temperature: 24±1° C., light-dark cycle: 12 hours/12 hours, illumination time: 7:00 a.m. to 7:00 p.m., relative humidity: 70% to 85%. The indoor environment was kept quiet and the mice could eat and drink freely. The tests were carried out at 1:00 to 5:00 in the afternoon. Before the test, the mice entered the experimental environment in advance for adaptation and were touched by the experimental personnel for adaptation.

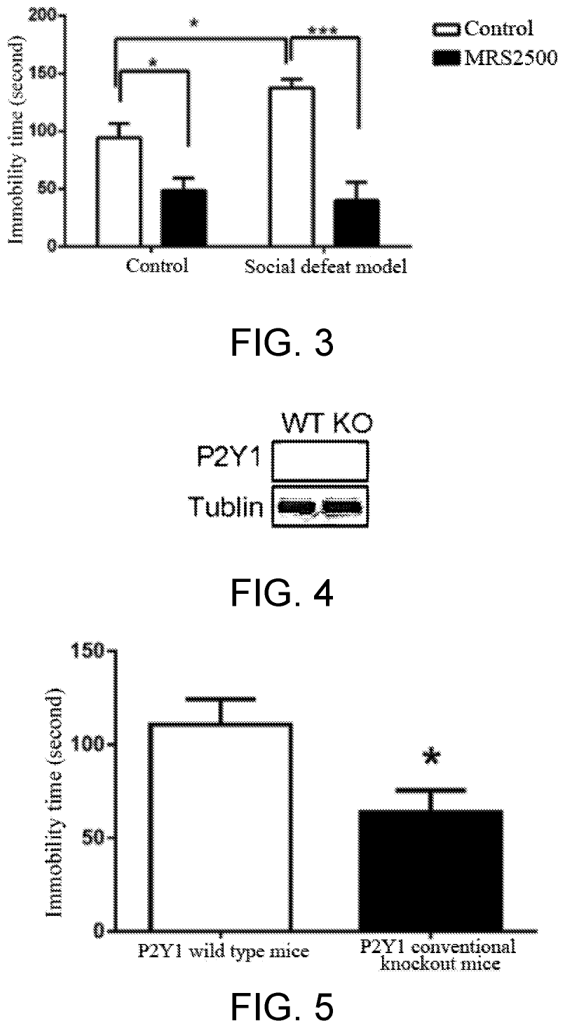

Test result: as shown in FIG. 3, the test group mice of the artificial cerebrospinal fluid group has significant reduced dwell duration at the interaction area with respect to the control group mice of the artificial cerebrospinal fluid group, indicating success modeling. Intracerebral perfusion of MRS2500 can improve the dwell duration at the interaction area of the test group mice. The result shows that after specific blocking of the P2Y1 receptor, an anti-depression effect was shown.

### Embodiment 2 Screening for Depression-Like Behavior of P2Y1 Receptor Conventional Knockout Mice

1. Forced Swimming Test

Test method: the P2Y1 receptor conventional knockout mice and wild-type mice were subjected to the forced swimming test for 6 minutes, and immobility time of swimming within the last 4 minutes was recorded.

Experimental animals: P2Y1 mice were purchased from Jax (strain number: 009131). The gene only has one coding exon, and most of the coding regions were interfered by means of the PGK neo cassette. The obtained 129 chimeric mice backcrossed with the C57BL/6J mice for 12 generations. The experimental animals were fed in an environment of 12 hours/12 hour light-dark cycle, and ate and drank freely.

Test result: as shown in FIG. 5, compared with the wild type mice, the P2Y1 receptor conventional knockout mice has significantly reduced immobility time during the forced swimming test. The result indicates that after the expression level of P2Y1 receptor was lowed, the mice showed anti-depression-like behavior.

2. Tail Suspension Test

A MED Associates tail suspension device was used in the test to real-time monitor motion state of the mice. The tail suspension box was open at the front side and closed at the rest of other sides. A pressure sensor was mounted at the bottom of the tail suspension rod, and the motion state of the mice was input to the computer via the pressure sensor for data process. A part 2 cm from the tail tip of the mouse was fixed and suspended within the tail suspension box (ENV-505TS, Med associates Inc.), allowing the mouse to be hung upside down and keeping the head approximately 10 cm away from the bottom of the box. At the beginning, the mice struggled intensively, and after finding out that there's no way to get rid of, the mice were in discontinuous immobile state, that is, the mice stopped struggling or passively and slightly swung. Software Med Suspension Tail automatically recorded, analyzed and processed the immobility time of the mice within 6 minutes, and the immobility time lower than a predetermined lower threshold was recorded as an index for testing depression-like behavior.

Experimental animals: P2Y1 mice were purchased from Jax (strain number: 009131). The gene only has one coding exon, and most of the coding regions were interfered by means of the PGK neo cassette. The obtained 129 chimeric mice backcrossed with the C57BL/6J mice for 12 generations. The experimental animals were fed in an environment of 12 hours/12 hours light-dark cycle, and ate and drank freely.

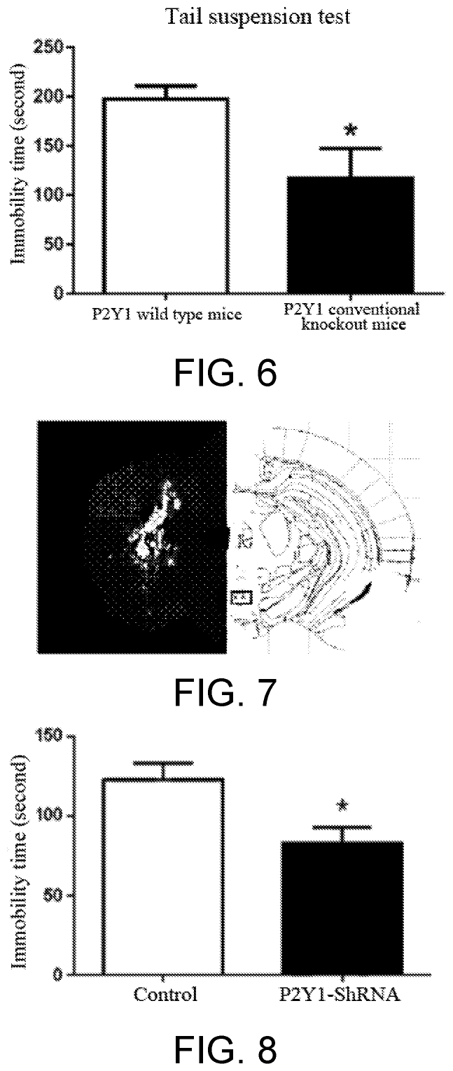

Test result: as shown in FIG. 6, compared with the wild type mice, the P2Y1 receptor conventional knockout mice has significantly reduced immobility time during the tail suspension test. The result indicates that after the expression level of P2Y1 receptor was lowed, the mice showed anti-depression-like behavior.

### Embodiment 3 Test of Verification of P2Y1 Receptor as a new Target for Anti-Depression by using RNA Interference Technology

Experimental animals: C57BL/6J mice, grade SPF, male, body weight of 20±2 g. The animals were all provided by the Laboratory Animal Center of Southern Medical University (Guangzhou, China). The mice were put in the SPF grade animal feeding room for feeding in separate cages, with 3 to 4 mice in each cage. Room temperature: 24±1° C., light-dark cycle: 12 hours/12 hours, illumination time: 7:00 a.m. to 7:00 p.m., relative humidity: 70% to 85%. The indoor environment was kept quiet and the mice could eat and drink freely. The tests were carried out at 1:00 to 5:00 in the afternoon. Before the test, the mice entered the experimental environment in advance for adaptation and were touched by the experimental personnel for adaptation.

Test method: the animal in each group was micro-injected with 0.5 μL of interfering RNA adeno-associated virus (shRNA) at the ventral tegmental area (angle: 7°, AP: −3.2 mm, ML: 1.0 mm, DV: −4.6 mm) (0.1 μL/min, remaining the needle for 5 minutes after injection), fed for 14 days and subjected to a forced swimming test after the expression recovery of virus. Titer of the P2Y1 specific-interfering shRNA is 1×10¹³ v.g/ml, 9.55E+08 copy/μL. Adeno-associated virus type 9 was used to package the virus, with the promoter being U6 and the serotype being AAV9 (packaged by Biowit Company, Shenzhen, China). Sequence information: the forward sequence is shown as SEQ ID NO:1; and the reverse sequence is shown as SEQ ID NO:2.

Test result: 14 days after the C57BL/6J mice were subjected to injection of adeno-associated virus, a large amount of nerve cells were infected by the shRNA virus, such that the ventral tegmental area expressed green fluorescent protein. It was found by using a laser scanning confocal microscope that shRNA mainly expressed at the ventral tegmental area of the mice. It was found from the result that with respect to the mice in the control virus group, the P2Y1-shRNA mice had significantly reduced immobility time during the forced swimming test, indicating that after the P2Y1 receptor was blocked, an anti-depression effect was shown.

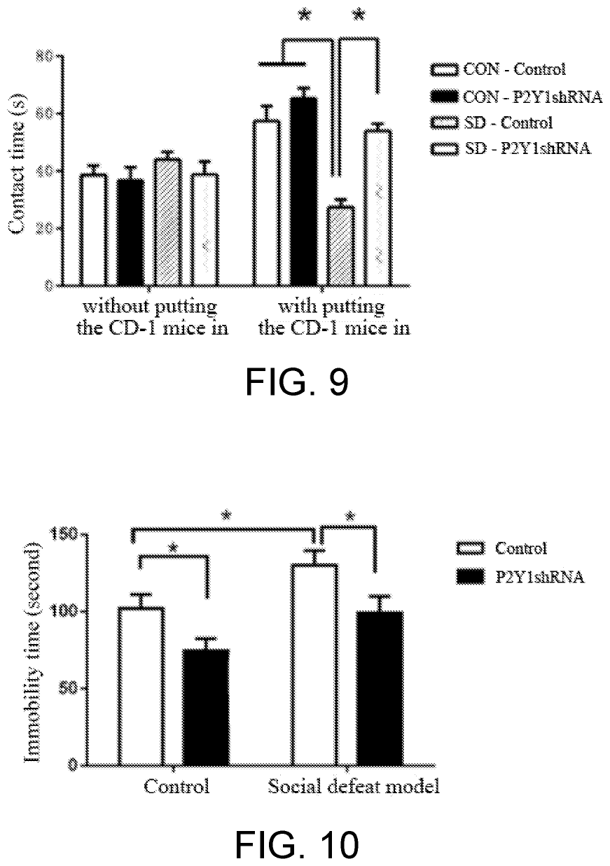

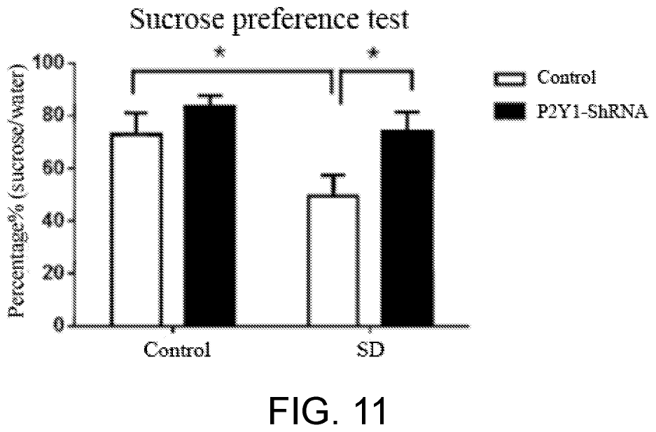

After modeling, the chronic social defeat stress depressed mice had one-day rest, and 14 days after the injection of shRNA virus, the depressed mice were checked whether the depressive situation had changed or not. In the social interaction test, the injection of shRNA could enhance the duration at the interaction area for the depressed mice, showing anti-depression-like behavior. In the forced swimming test, the injection of shRNA could reduce the increased immobility time of the depressed mice, showing anti-depression-like behavior. In the sucrose preference test, the injection of shRNA could enhance the decreased sucrose preference of the depressed mice. Such results further prove that blocking of P2Y1 receptor has an anti-depression effect (as shown in FIG. 7 to FIG. 11).

With reference to the disclosure and teaching of the above description, those skilled in the art of the present invention can still make suitable modification and amendments to the above-mentioned implementations. Therefore, the present invention is not limited to the above disclosure and the described specific implementations, and some amendments and modifications made to the present invention shall all fall within the scope of protection of the claims in the present invention. In addition, though some specific terms are used in the description, these terms are merely for convenience of explanation and do not limit the present invention in any way.

1. A P2Y1 receptor, wherein the P2Y1 receptor is used as a target in screening for a medicine for preventing and treating depression and/or anxiety disorder, as a biomarker in screening for depression and/or anxiety disorder and in early warning of depression and/or anxiety disorder, or in screening for clinical diagnosis reagents for depression and/or anxiety disorder.

2. (canceled)

3. (canceled)

4. An antagonist of P2Y1 receptor, wherein the antagonist of P2Y1 receptor is used in anti-depression and/or anti-anxiety disorder.

5. The antagonist of P2Y1 receptor according to claim 4, wherein the antagonist of P2Y1 receptor is a compound or polypeptide inhibiting the P2Y1 receptor.

6. The antagonist of P2Y1 receptor according to claim 5, wherein the antagonist of P2Y1 receptor is MRS2179 and derivatives thereof, or MRS2500 and derivatives thereof.

7. A medicine of anti-depression and/or anti-anxiety disorder, comprising an effective dosage of an antagonist of P2Y1 receptor, and a pharmaceutically acceptable carrier.

8. The medicine of anti-depression and/or anti-anxiety disorder according to claim 7, wherein the antagonist of P2Y1 receptor is a compound or polypeptide inhibiting the P2Y1 receptor.

9. The medicine of anti-depression and/or anti-anxiety disorder according to claim 8, wherein the antagonist of P2Y1 receptor is MRS2179 and derivatives thereof, or MRS2500 and derivatives thereof.

10. The medicine of anti-depression and/or anti-anxiety disorder according to claim 7, wherein the medicine has a dosage form of liquid or solid.
